(19) United States
(12) Patent Application Publication
(10) Pub. No.: US 2022/0211610 A1
(43) Pub. Date: Jul. 7, 2022

(54) SKIN WHITENING COSMETIC COMPOSITION COMPRISING LACTOBACILLUS RHAMNOSUS LM1011 HAVING IMMUNOSTIMULATING ACTIVITY

(71) Applicant: LACTOMASON CO., LTD., Jinju-si Gyeongsangnam-do (KR)

(72) Inventors: Min Gyu JEON, Jinju-si (KR); Ji Yeon LEE, Jinju-si (KR); Do Seon HONG, Seoul (KR)

(21) Appl. No.: 17/605,468

(22) PCT Filed: Apr. 24, 2020
(86) PCT No.: PCT/KR2020/005462

§ 371 Date: Oct. 21, 2021

(30) Foreign Application Priority Data
Apr. 26, 2019 (KR) 10-2019-0049189

(51) Int. Cl. A61K 8/99; A61Q 19/02

(52) U.S. Cl. CPC A61K 8/99; A61K 2800/782; A61Q 19/02

(57) ABSTRACT

The present disclosure relates to a skin whitening cosmetic composition comprising Lactobacillus rhamnosus LM1011 having immunostimulating activity, and more particularly provides a skin whitening cosmetic composition comprising Lactobacillus rhamnosus LM1011, which reduces melanin production by regulating tyrosinase activity, as an active ingredient.

### TECHNICAL FIELD

The present disclosure relates to a skin whitening cosmetic composition comprising Lactobacillus rhamnosus LM1011 having immunostimulating activity, and more particularly, to a skin whitening cosmetic composition comprising Lactobacillus rhamnosus LM1011, which reduces melanin production by regulating tyrosinase activity, as an active ingredient.

### BACKGROUND

Human skin color is determined mainly by the content of pigment called melanin in skin. Melanin is biosynthesized by melanocytes present in the basal layer of the epidermis and transferred from the basal layer of the epidermis to the keratin layer through cytoplasmic processes by keratinization of keratinocytes.

Melanin is produced through various oxidation processes after tyrosine, which is a kind of amino acid, is converted into DOPA and DOPA quinone by an enzyme called tyrosinase in melanocytes. Melanin absorbs ultraviolet (UV) light and protects various cellular tissues present inside the epidermis from the damaging effects of UV light. Excessive synthesis of melanin causes freckles to appear and darkens the skin color. Therefore, if the excessive synthesis of melanin is inhibited, it is possible to brighten the skin color and reduce skin hyperpigmentation such as freckles.

Meanwhile, Lactobacillus rhamnosus is a kind of lactic acid bacteria and is known to have various effects, such as intestinal regulation, antibacterial action, atopy prevention and body fat reduction. Also, in recent years, a lot of research on killed Lactobacillus has been conducted. As for the killed Lactobacillus, it is known that lipoteichoic acid, which is a cell wall component generated in the process of killing live bacteria, adheres to the intestinal wall to inhibit the settlement of harmful bacteria and antibacterial components, such as lactic acid and bacteriocin, in fermented products can directly act on and inhibit harmful bacteria. Also, it is found that immunoglobulin A, which is important in defense against infection of external bacteria, is produced generally when killed Lactobacillus rather than live Lactobacillus is absorbed through small intestine and stimulate macrophages and the killed Lactobacillus is used as food for live bacteria and functions as excellent prebiotics in the intestinal regulation of Lactobacillus.

### DISCLOSURE OF THE INVENTION

### Problems to be Solved by the Invention

The present disclosure is conceived to provide a skin whitening cosmetic composition comprising Lactobacillus rhamnosus LM1011 having immunostimulating activity, which reduces melanin production by regulating tyrosinase activity, as an active ingredient.

However, problems to be solved by the present disclosure are not limited to the above-described problems. Although not described herein, other problems to be solved by the present disclosure can be clearly understood by a person with ordinary skill in the art from the following description.

### Means for Solving the Problems

A first aspect of the present disclosure provides a skin whitening cosmetic composition comprising Lactobacillus rhamnosus LM1011 (Depository Institution: Korean Collection for Type Cultures, Accession Number: KCTC12751BP, Date of Deposit: Apr. 18, 2019) having immunostimulating activity.

### Effects of the Invention

A skin whitening cosmetic composition comprising Lactobacillus rhamnosus LM1011 (Depository Institution: Korean Collection for Type Cultures, Accession Number: KCTC12751BP, Date of Deposit: Apr. 18, 2019) having immunostimulating activity according to an embodiment of the present disclosure reduces the production of melanin by regulating tyrosinase activity and thus has a skin whitening effect against, particularly, hypermelanosis.

### BRIEF DESCRIPTION OF THE DRAWINGS

FIG. 1 is a schematic diagram showing the mechanism of reducing melanin production of Lactobacillus rhamnosus LM1011 (Depository Institution: Korean Collection for Type Cultures, Accession Number: KCTC12751BP, Date of Deposit: Apr. 18, 2019) according to an example of the present disclosure.

FIG. 2 is a graph showing the effect of reducing melanin production by a skin whitening cosmetic composition comprising Lactobacillus rhamnosus LM1011 according to an example of the present disclosure in a B16-F10 melanoma cell line.

FIG. 3 is a graph showing changes in improvement rate of M-index (melanin index) by a skin whitening cosmetic composition comprising Lactobacillus rhamnosus LM1011 over time measured using a Mexameter® in a clinical test on the skin whitening effect on hypermelanosis.

FIG. 4 is a graph showing changes in visual evaluation score of a skin whitening cosmetic composition comprising Lactobacillus rhamnosus LM1011 over time in a clinical test on the skin whitening effect on hypermelanosis according to an example of the present disclosure.

FIG. 5 is a graph showing changes in improvement rate of L-index (brightness index) over time measured using a chromameter in a clinical test on the skin whitening effect on hypermelanosis according to an example of the present disclosure.

FIG. 6 shows whether Lactobacillus rhamnosus LM1011 is cytotoxic according to an example of the present disclosure.

FIG. 7 shows whether Lactobacillus rhamnosus HK-9 is cytotoxic according to an example of the present disclosure.

FIG. 8 shows the effect of inhibiting the expression of tyrosinase when Lactobacillus rhamnosus LM1011 and Lactobacillus rhamnosus HK-9 are treated with an alpha melanocyte stimulating hormone (α-MSH) according to an example of the present disclosure.

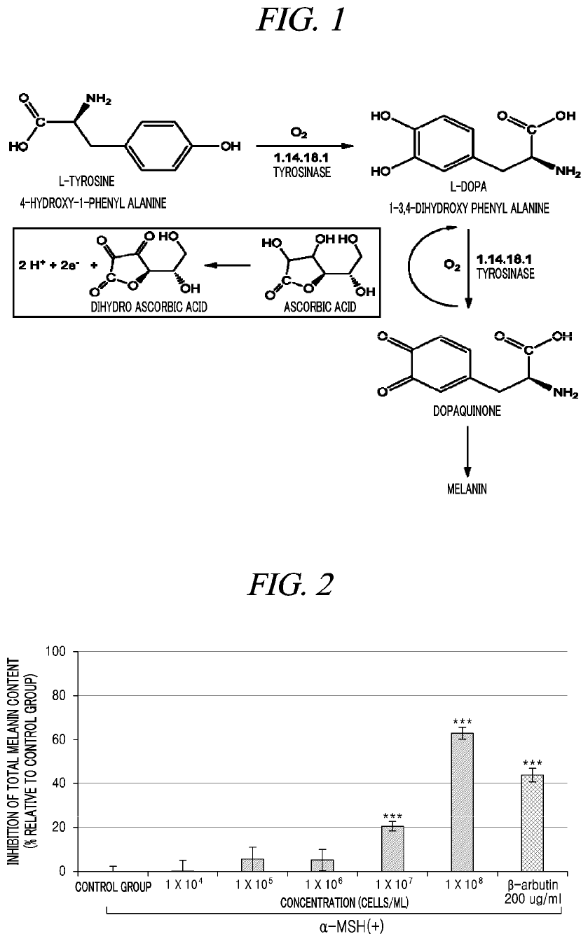

### BEST MODE FOR CARRYING OUT THE INVENTION

Hereinafter, embodiments of the present disclosure will be described in detail with reference to the accompanying drawings so that the present disclosure may be readily implemented by a person with ordinary skill in the art. However, it is to be noted that the present disclosure is not limited to the embodiments but can be embodied in various other ways. In drawings, parts irrelevant to the description are omitted for the simplicity of explanation, and like reference numerals denote like parts through the whole document.

Through the whole document, the term “connected to” or “coupled to” that is used to designate a connection or coupling of one element to another element includes both a case that an element is “directly connected or coupled to” another element and a case that an element is “electronically connected or coupled to” another element via still another element.

Through the whole document, the term “on” that is used to designate a position of one element with respect to another element includes both a case that the one element is adjacent to the other element and a case that any other element exists between these two elements.

Through the whole document, the term “comprises or includes” and/or “comprising or including” used in the document means that one or more other components, steps, operation and/or existence or addition of elements are not excluded in addition to the described components, steps, operation and/or elements unless context dictates otherwise. Through the whole document, the term “about or approximately” or “substantially” is intended to have meanings close to numerical values or ranges specified with an allowable error and intended to prevent accurate or absolute numerical values disclosed for understanding of the present disclosure from being illegally or unfairly used by any unconscionable third party. Through the whole document, the term “step of” does not mean “step for”.

Through the whole document, the term “combination(s) of” included in Markush type description means mixture or combination of one or more components, steps, operations and/or elements selected from a group consisting of components, steps, operation and/or elements described in Markush type and thereby means that the disclosure includes one or more components, steps, operations and/or elements selected from the Markush group.

Through the whole document, a phrase in the form “A and/or B” means “A or B, or A and B”.

Hereinafter, embodiments and examples of the present disclosure will be described in detail with reference to the accompanying drawings. However, the present disclosure may not be limited to the following embodiments, examples, and drawings.

A first aspect of the present disclosure provides a skin whitening cosmetic composition comprising Lactobacillus rhamnosus LM1011 (Depository Institution: Korean Collection for Type Cultures, Accession Number: KCTC12751BP, Date of Deposit: Apr. 18, 2019) having immunostimulating activity.

In an embodiment of the present disclosure, the skin whitening cosmetic composition comprising Lactobacillus rhamnosus LM1011, a killed bacterial cell thereof, a crush thereof, a culture product thereof, a concentrate thereof or an extract thereof as an active ingredient, but may not be limited thereto. For example, the skin whitening cosmetic composition may contain a heat-treated Lactobacillus rhamnosus LM1011 strain, but may not be limited thereto.

In an embodiment of the present disclosure, the Lactobacillus rhamnosus LM1011 strain is heat treated so that killed Lactobacillus cells themselves can be used and when the Lactobacillus rhamnosus LM1011 strain is applied to a cosmetic product, product instability caused by abnormal fermentation can be reduced, but the present disclosure may not be limited thereto.

In an embodiment of the present disclosure, the skin whitening cosmetic composition is not particularly limited to the amount of Lactobacillus rhamnosus LM1011 as long as it contains Lactobacillus rhamnosus LM1011. For example, the skin whitening cosmetic composition may contain Lactobacillus rhamnosus LM1011 at a concentration of from about 1×10¹¹ cells/mL to about 5×10¹¹ cells/mL, but may not be limited thereto.

In an embodiment of the present disclosure, if the skin whitening cosmetic composition contains Lactobacillus rhamnosus LM1011 at a concentration of less than about 1×10¹¹ cells/mL or more than about 5×10¹¹ cells/mL, the skin whitening effect can be reduced. Therefore, the concentration of Lactobacillus rhamnosus LM1011 may be in the range of from about 1×10¹¹ cells/mL to about 5×10¹¹ cells/mL, from about 1×10¹¹ cells/mL to about 4×10¹¹ cells/mL, from about 1×10¹¹ cells/mL to about 3×10¹¹ cells/mL, from about 1×10¹¹ cells/mL to about 2×10¹¹ cells/mL, from about 2×10¹¹ cells/mL to about 5×10¹¹ cells/mL, or from about 3×10¹¹ cells/mL to about 5×10¹¹ cells/mL, but may not be limited thereto.

In an embodiment of the present disclosure, Lactobacillus rhamnosus LM1011 can reduce melanin production, but may not be limited thereto. For example, Lactobacillus rhamnosus LM1011 can reduce the production of melanin increased in the skin due to exposure to UV light, but may not be limited thereto.

In an embodiment of the present disclosure, Lactobacillus rhamnosus LM1011 can reduce melanin production by regulating tyrosinase activity, but may not be limited thereto. For example, Lactobacillus rhamnosus LM1011 converts L-tyrosine to L-dopa and converts the L-dopa to dopaquinone to regulate the activity of tyrosinase, which is an enzyme that produces melanin, and specifically to inhibit the expression level and/or activity of tyrosinase and thus can reduce the production of melanin in the skin, but may not be limited thereto.

In an embodiment of the present disclosure, the skin whitening cosmetic composition is not particularly limited to the formulation and may have any one formulation selected from, for example, toner, lotion, essence, cream, pack, foundation and makeup base, but may not be limited thereto.

In an embodiment of the present disclosure, the skin whitening cosmetic composition may further contain any ingredient selected according to the formulation or purpose of use of cosmetics, but may not be limited thereto. For example, the skin whitening cosmetic composition may further contain purified water, oils, surfactants, moisturizing agents, higher alcohols, thickeners, chelating agents, pigments, fatty acids, preservatives, waxes, pH regulators, fragrances and the like, but may not be limited thereto.

In an embodiment of the present disclosure, if the formulation of the skin whitening cosmetic composition is paste, cream or gel, animal and vegetable fats, oils, waxes, paraffins, starch, tragacanth, cellulose derivatives, polyethylene glycols, silicones, bentonites, silica, talc or zinc oxide may be used as the carrier ingredient, but the present disclosure may not be limited thereto.

In an embodiment of the present disclosure, if the formulation of the skin whitening cosmetic composition is powder or spray, lactose, talc, silica, aluminum hydroxide, calcium silicates or polyamide powder may be contained as the carrier ingredient, and particularly, in the case of spray, it may further contain a propellant such as chlorofluorohydrocarbon, propane/butane or dimethyl ester, but may not be limited thereto.

In an embodiment of the present disclosure, if the formulation of the skin whitening cosmetic composition is a solution or an emulsion, solvents, solvating agents or emulsifying agents may be used as the carrier ingredient, and examples thereof may include water, ethanol, isopropanol, ethyl carbonate, ethyl acetate, benzyl alcohol, benzyl benzoate, propylene glycol, 1,3-butylglycol oil, glycerol aliphatic ester, polyethylene glycol or sorbitan fatty acid ester, but may not be limited thereto.

In an embodiment of the present disclosure, if the formulation of the skin whitening cosmetic composition is a suspension, liquid diluting agents, such as water, ethanol or propylene glycol, suspending agents, such as ethoxylated isostearyl alcohol, polyoxyethylene sorbitol ester and polyoxyethylene sorbitan ester, microcrystalline cellulose, aluminum meta-hydroxide, bentonite, agar or tragacanth, etc. may be used as the carrier ingredient, but the present disclosure may not be limited thereto.

In an embodiment of the present disclosure, if the formulation of the skin whitening cosmetic composition is a surfactant-containing cleansing composition, aliphatic alcohol sulfate, aliphatic alcohol ether sulfate, sulfosucinnate monoester, isethionate, imidazolinium derivatives, methyltaurate, sarcocinate, fatty acid amide ether sulfate, alkylamido betaine, aliphatic alcohol, fatty acid glyceride, fatty acid diethanolamide, vegetable oils, lanoline derivatives or ethoxylated glycerol fatty acid ester may be used as the carrier ingredient, but the present disclosure may not be limited thereto.

A second aspect of the present disclosure relates to a skin improving method includes administering or applying a skin whitening cosmetic composition comprising Lactobacillus rhamnosus LM1011 (Depository Institution: Korean Collection for Type Cultures, Accession Number: KCTC12751BP, Date of Deposit: Apr. 18, 2019) as an active ingredient. The skin improving method includes skin whitening. The features described above in respect of the first aspect of may equally apply to the method according to the second aspect.

### MODE FOR CARRYING OUT THE INVENTION

Hereinafter, the present disclosure will be explained in more detail with reference to Examples. However, the following Examples are illustrative only for better understanding of the present disclosure but do not limit the present disclosure.

### Examples

1. Preparation of Killed Lactobacillus Cell and Cell Test

1-1. Preparation of Skin Whitening Cosmetic Composition Comprising Lactobacillus rhamnosus LM1011

According to the present example, killed Lactobacillus rhamnosus LM1011 cells were prepared by batch cultivation.

The Lactobacillus was cultured in 60 L of culture medium in a 100 L fermenter (KoBio Tech) at a stir rate of 30 rpm and a temperature of from 35° C. to 37° C., and pH was maintained at 5 to 6.5. After culture, the culture medium was centrifuged at 13,000 rpm for 30 minutes to collect bacterial cells. The collected bacterial cells were diluted twice compared to the weight by using physiological saline (0.9% NaCl) and tyndallized for 10 minutes at 110° C. in an autoclave, followed by lyophilization to finally obtain heat-treated Lactobacillus rhamnosus LM1011.

1-2. Effect of Reducing Melanin Production (In Vitro)

The effect of reducing melanin production by the heat-treated Lactobacillus rhamnosus LM1011 obtained according to the above-described example was checked. A B16-F10 melanoma cell line was used as a cell line and inoculated at a concentration of 1×10⁴ cells/well into a 6-well cell culture plate. After culture for 24 hours, the cell line was treated with the heat-treated Lactobacillus rhamnosus LM1011 at a concentration of 0 (negative control group), 1×10⁴, 1×10⁵, 1×10⁶, 1×10⁷ and 1×10⁸ cells/mL and then cultured for 48 hours. The inhibition of melanin production was checked by measuring the amount of protein expressed in the cells using an EX-CYTOX (EZ-3000, Daeil lab service) and absorbance at 450 nm with a multi-plate reader and then comparing the melanin synthesis inhibition rate with the negative control group. Besides the heat-treated Lactobacillus rhamnosus LM1011 strain test groups, 200 ug/mL of β-albumin, which is a compound that has a skin whitening function, was also compared as a positive control group.

As shown in FIG. 2, the heat-treated Lactobacillus rhamnosus LM1011 showed the effective effect of reducing melanin production at a concentration of from 1×10⁵ to 1×10⁸ cells/mL compared to the negative control group. Particularly, the heat-treated Lactobacillus rhamnosus LM1011 showed about 20% higher effect of reducing melanin production at a concentration of 1×10⁸ cells/mL than β-albumin.

2. Preparation of Killed Bacterial Cell-Containing Cosmetics and Human In Vivo Test

2-1. Preparation of Cosmetics

The killed Lactobacillus rhamnosus LM1011 cells prepared according to the above example were diluted twice to less than ten times compared to the weight to adjust the concentration of cells to 1×10¹¹ cells/mL.

A control serum group and a test serum group were prepared by a typical method in the blend amount as shown in Table 1.

TABLE 1

| Ingredient | Blend amount (%) | Control group | Test group |
|---|---|---|---|
| Killed bacterial cells | 3 | X | ◯ |
| Polyethylene glycol | 8 | ◯ | ◯ |
| Ethyl hexyl glycerin | 0.1 | ◯ | ◯ |
| Butylene glycol | 0.25 | ◯ | ◯ |
| Purified water | Rest amount | ◯ | ◯ |

2-2. Skin Whitening Effect on Hypermelanosis (In Vivo)

The skin whitening effect of the skin whitening cosmetic composition comprising the heat-treated Lactobacillus rhamnosus LM1011 of the above example as an active ingredient was checked through a clinical test. The test was conducted on 23 subjects with hypermelanosis that met the selection criteria but did not meet the exemption criteria, and adopted twice a day self-application. The skin whitening effect was checked through M-index (hyperpigmentation improvement index) evaluation using a Mexameter®, survey on the subjects, cutaneous irritation evaluation by dermatologists and visual evaluation by dermatologists.

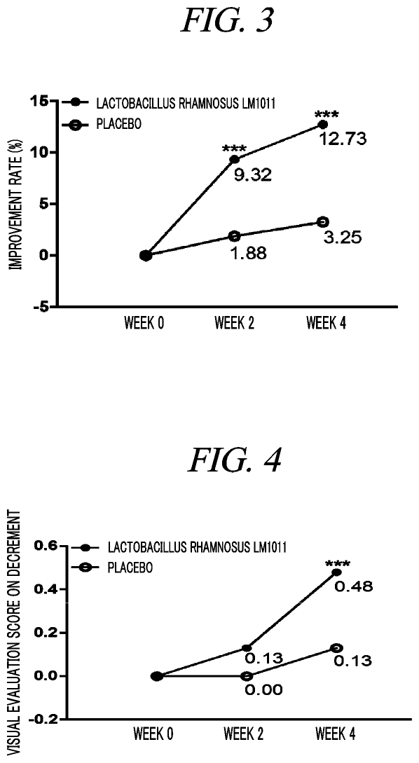

Referring to FIG. 3, each improvement rate of hyperpigmentation by the skin whitening cosmetic composition was obtained by obtaining the differences in the M-index before and after the test between a control area and a test area, and FIG. 3 shows improvement rates over time in percentage relative to the measured values before the test. Compared to a placebo group, which is a control group, the skin whitening cosmetic composition of the present disclosure showed a significant increase in the improvement rate of hyperpigmentation over time after application to the skin.

Referring to FIG. 4, each visual evaluation score of the skin whitening cosmetic composition was obtained by obtaining the differences in value before and after the test between a control area and a test area. Compared to a placebo group, which is a control group, the skin whitening cosmetic composition of the present disclosure showed an increase in pigment reduction over time.

The above-described results demonstrate that the skin whitening cosmetic composition comprising the heat-treated Lactobacillus rhamnosus LM1011 of the present disclosure as an active ingredient has melanin reducing effect and skin whitening effect when applied to the skin.

2-3. Brightening Effect (In Vivo)

The skin whitening effect of the skin whitening cosmetic composition comprising the heat-treated Lactobacillus rhamnosus LM1011 of the above example as an active ingredient was checked through a clinical test. The test was conducted on 23 subjects that met the selection criteria but did not meet the exemption criteria, and adopted twice a day self-application. The brightening effect was expressed in skin brightness index improvement rate by measuring a facial brightness value (L-index) with a chromameter.

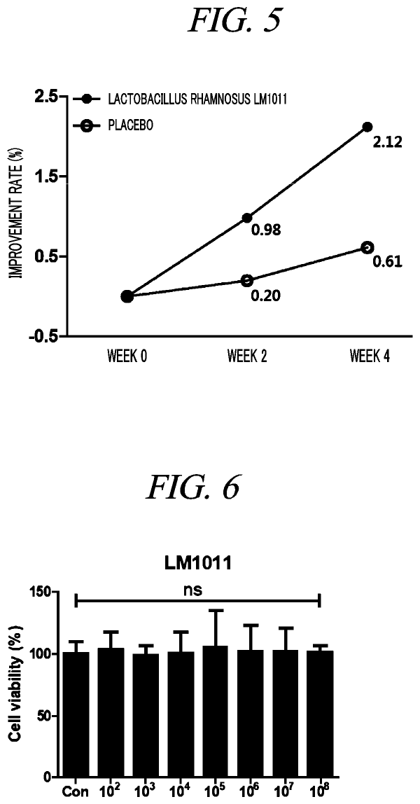

Referring to FIG. 5, the brightening effect of the skin whitening cosmetic composition was obtained by obtaining the differences in the L-index before and after the test between a control area and a test area, and FIG. 5 shows improvement rates over time in percentage relative to the measured values before the test. Compared to a placebo group, which is a control group, the skin whitening cosmetic composition of the present disclosure showed a significant increase in the skin brightness improvement rate over time after use. The above-described result demonstrates that the skin whitening cosmetic composition comprising the heat-treated Lactobacillus rhamnosus LM1011 of the present disclosure as an active ingredient has brightening effect when applied to the skin.

3. Evaluation of Tyrosinase Inhibition Activity of Lactobacillus rhamnosus LM1011

3-1. Cytotoxicity Check

To compare and check inhibition activity of Lactobacillus rhamnosus LM1011 according to the present example on tyrosinase, which is a key enzyme regulating melanin production, with that of Lactobacillus rhamnosus HK-9 (hereinafter, referred to as HK-9), the following test was conducted.

First, in order to check the cytotoxicity of LM1011 and HK-9 and determine the treatment concentration, B16F10 cells were inoculated at a concentration of 5×10⁴ cells/well into a 96-well plate. After stabilization for 24 hours, the B16F10 cells were treated with Lactobacillus at a concentration of 1×10² to 1×10⁸ cells/ml. After 24 hours, the B16F10 cells were treated with a mixture of 95 μl of DMEM (gibco) and 5 μl of Cell Counting Kit-8 (CCK-8, Woongbee Meditech Co.). After 2 hours, measurement was performed with a multi plate reader (450 nm) and the measured values were converted into percentage based on the control group to compare cytotoxicity.

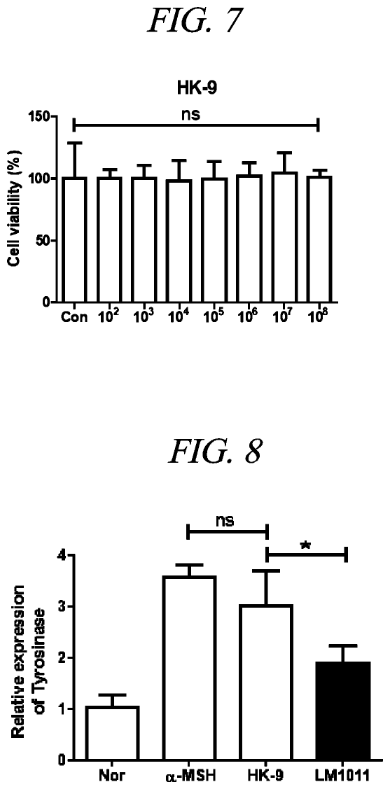

According to the results presented in FIG. 6 and FIG. 7, it was confirmed that there was no statistical difference in cytotoxicity depending on the treatment concentration of LM1011 and HK-9. For the subsequent test, the treatment concentration of LM1011 and HK-9 was determined to be 1×10⁸ cells/ml.

3-2. Evaluation of Tyrosinase Expression Inhibition

To evaluate the effect of inhibiting the expression of tyrosinase when Lactobacillus rhamnosus LM1011 and Lactobacillus rhamnosus HK-9 are treated with an alpha melanocyte stimulating hormone (α-MSH), the following test was conducted.

First, a B16F10 cell line was inoculated at a concentration of 2×10⁵ cells/well into a 6-well plate. After stabilization for 24 hours, the test was conducted to a non-treatment group (Nor), an α-MSH treatment group, an α-MSH+HK-9 treatment group and an α-MSH+LM1011 treatment group. After treatment for 24 hours, the medium was removed, washed with PBS and then treated with 500 μl of Trizol to crush the cells. The cells were crushed sufficiently and then treated with 100 μl of chloroform and centrifuged (13,000 rpm, 15 minutes, 4° C.) to separate the cell debris and RNA. Then, 240 μl of the supernatant was taken and treated with an equal volume of isopropyl alcohol (IPA) to allow the RNA to be precipitated, followed by centrifugation (13,000 rpm, 15 minutes, 4° C.). The supernatant was removed, followed by treatment with 700 μl of 75% ethanol (13,000 rpm, 5 minutes, 4° C.) and centrifugation. After sufficient drying, the RNA was dissolved in 30 μl of ultra pure water.

Then, after 1 μg of the separated RNA was taken to synthesize cDNA using a iScript cDNA synthesis kit (BIO-RAD). A PCR reaction was performed as shown in Table 2 below.

TABLE 2

| Reaction stage | Reaction conditions |
|---|---|
| Priming | 5 min at 25° C. |
| Reverse transcription | 20 min at 46° C. |
| RT inactivation | 1 min at 95° C. |
| Optional step | Hold at 4° C. |

After the synthesis of cDNA was completed, the amount of tyrosinase expression was evaluated through real-time PCR (viia 7, Applied Biosystems) with a TaqMan probe (Tyrosinase, Mm00495817_m1, Applied Biosystems) using 260 ng of cDNA as a template. As a result, it was confirmed that the expression pattern of tyrosinase under stimulation of α-MSH was significantly decreased in an LM1011 treatment group compared to an HK-9 treatment group (FIG. 8, *p<0.05).

According to the above results, it was confirmed that LM1011 of the present disclosure more significantly inhibited the expression of tyrosinase caused by stimulation of melanocyte stimulating hormone at concentrations without cytotoxicity than HK-9, which is a Lactobacillus rhamnosus strain. The effect of inhibiting melanin synthesis in the skin by LM1011 of the present disclosure is considered to be significantly superior to the existing Lactobacillus rhamnosus strains. Therefore, the LM1011 strain of the present disclosure is a composition for skin whitening and can be used as a functional material applicable to cosmetics, foods, etc.

The above description of the present disclosure is provided for the purpose of illustration, and it would be understood by a person with ordinary skill in the art that various changes and modifications may be made without changing technical conception and essential features of the present disclosure. Thus, it is clear that the above-described examples are illustrative in all aspects and do not limit the present disclosure. For example, each component described to be of a single type can be implemented in a distributed manner. Likewise, components described to be distributed can be implemented in a combined manner.

The scope of the present disclosure is defined by the following claims rather than by the detailed description of the embodiment. It shall be understood that all modifications and embodiments conceived from the meaning and scope of the claims and their equivalents are included in the scope of the present disclosure.

### ACCESSION NUMBER

Depository Institution: Korea Research Institute of Bioscience and Biotechnology

Accession Number: KCTC12751BP

Date of Deposit: Apr. 18, 2019

We claim:

1. A skin whitening cosmetic composition comprising Lactobacillus rhamnosus LM1011 as an active ingredient.

2. The skin whitening cosmetic composition of claim 1,
wherein the skin whitening cosmetic composition comprises the Lactobacillus rhamnosus LM1011 at a concentration of from 1×10¹¹ cells/mL to 5×10¹¹ cells/mL.

3. The skin whitening cosmetic composition of claim 1,
wherein the Lactobacillus rhamnosus LM1011 reduces melanin production.

4. The skin whitening cosmetic composition of claim 3,
wherein the Lactobacillus rhamnosus LM1011 reduces melanin production by regulating tyrosinase activity.

5. The skin whitening cosmetic composition of claim 1,
wherein the skin whitening cosmetic composition has any one formulation selected from toner, lotion, essence, cream, mask pack, foundation and makeup base.
